(19) United States
(12) Patent Application Publication
(10) Pub. No.: US 2023/0097608 A1
(43) Pub. Date: Mar. 30, 2023

(54) SYSTEM FOR ESTABLISHING AN INSTANT COMMUNICATION SESSION WITH A HEALTH PROVIDER

(71) Applicant: Evernorth Strategic Development, Inc., St. Louis, MO (US)

(72) Inventors: Rahib Diwan, Irvine, CA (US); Gang Wu, Ballwin, MO (US); Christopher G. Lehmuth, St. Louis, MO (US)

(21) Appl. No.: 17/489,067

(22) Filed: Sep. 29, 2021

(51) Int. Cl. G16H 40/20; G06Q 10/06; G16H 50/30; G16H 20/70; G16H 80/00; G06N 20/00

(52) U.S. Cl. CPC G16H 40/20; G06Q 10/063112; G16H 50/30; G16H 20/70; G16H 80/00; G06N 20/00

(57) ABSTRACT

A system and method of establishing instant communication session with health providers starts with processor receiving request from member-related client device and generating, using a topic model engine, keywords based on reported information associated with user of the member-related client device and historical provider information associated with user. Processor generates risk scores using predictive models associated with conditions and identifies specialists based on the keywords and the risk scores. Processor selects a subset of the specialists based on a user's preferences associated with gender, age, or language and identifies a specialist match based on a highest satisfaction score or a lowest contractual rate. The specialist match is a specialist included in the subset. Processor establishes a communication session between the member-related client device and a provider client device associated with the specialist match. Other embodiments are disclosed herein.

### BACKGROUND

When a customer needs to communicate with a licensed behavioral or mental health specialist, the need is usually urgent and time-sensitive. Current customer service systems are cumbersome and time-consuming.

For example, hotlines for suicide prevention or domestic abuse support require that the customer have prior knowledge of the telephone number and are also limited to voice interactions. Other customer service systems can provide an asynchronous interaction with a specialist where the customer schedules a communication session at a later time. These asynchronous solutions do not address the customer's immediate need to access the specialist such that the patient's mental and physical well-being can be negatively impacted.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some embodiments are illustrated by way of example, and not limitation, in the figures of the accompanying drawings in which:

FIG. 1 is a block diagram showing an example system 100 including a connection system 108 according to various exemplary embodiments.

FIG. 2 is block diagram illustrating further details regarding the connection system 108, according to exemplary embodiments.

FIG. 3 is a schematic diagram illustrating data which may be stored in the database 208 of the connection system 108, according to various exemplary embodiments.

FIG. 4 illustrates a process 400 of establishing an instant communication session with a health provider in accordance with one embodiment.

FIG. 5 illustrates a user interface of a member-related client device 104 displaying a selectable item 502 in accordance with one embodiment.

FIG. 6 is a block diagram showing a software architecture within which examples may be implemented.

FIG. 7 is a diagrammatic representation of a specific dedicated machine in the form of a computer system within which a set of instructions may be executed for causing the machine to perform any one or more of the methodologies discussed herein, in accordance with some examples.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

Among other things, embodiments of the present disclosure improve the functionality of customer service methods and customer service electronic systems. An organization can provide its customers or members with access to customer service via different communication channels including audio calls (e.g., telephone call, VoIP, audio message, etc.) or via electronic messages (e.g., online chat, instant messaging, email, etc.) using the customer service methods and the customer service electronic systems.

When a user or customer needs to communicate with a licensed behavioral or mental health specialist, the need is usually urgent and time-sensitive. Embodiments of the present disclosure improve the functionality of customer service methods and systems by providing the user with nearly instant access to the health specialist via a selectable item (e.g., link, icon, image, etc.) that is displayed on the user's client device. The user is able to obtain access to the health specialist using the user's preferred communication method (e.g., chat, video call, or audio call) by activating the selectable item via a single click or touch (e.g., one touch activation). Embodiments of the present disclosure selects the health specialist best suited to help the user and establishes a communication session between the user's client device and the health specialist's client device. Embodiments of the present disclosure further organizes the selectable item on the user's electronic device showing multiple (e.g., all) types of connections and a subset of the types of connections that are preferred for the user or predicted to be better for the user by the system.

### Networked Computing Environment

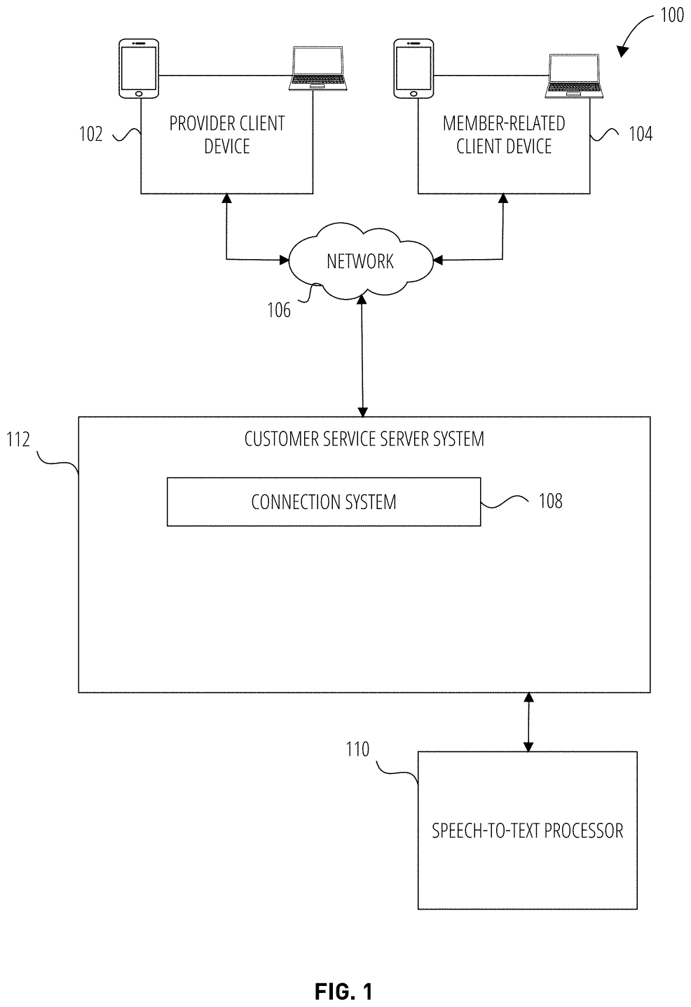

FIG. 1 is a block diagram showing an example system 100 according to various exemplary embodiments. The system 100 can be a customer service system that includes a member-related client device 104, a provider client device 102, and a member-related client device 104 that are communicatively coupled over a network 106 (e.g., Internet, telephony network, electronic communication network or the like).

The provider client device 102 and the member-related client device 104 can be communicatively coupled via an audio call (e.g., VoIP, Public Switched Telephone Network, cellular communication network, etc.), via electronic messages (e.g., online chat, instant messaging, text messaging, email, and the like), or via a video call (e.g., video teleconferencing via Internet or cellular communication network). In another embodiment, the provider client device 102 and the member-related client device 104 are communicatively coupled via a voice call, e.g., using a telephony network 106. While FIG. 1 illustrates a single provider client device 102 and a single member-related client device 104, it is understood that a plurality of provider client devices 102 and a plurality of member-related client devices 104 can be included in the system 100 in other embodiments. As used herein, the term “client device” may refer to any machine that interfaces to a communications network (such as network 106) to obtain resources from one or more server systems or other client devices. A client device may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network. The member-related client device 104 can include a microphone and speaker on a mobile electronic device, a telephone, or a self-service kiosk, e.g., at a pharmacy, a clinic, a doctor's office, a mobile relief center, and the like. The member-related client device 104 can also include telecommunications devices for the deaf (TDD). The member-related client device 104 can load instructions from its memory or from a remote server and, at times, be a dedicated machine providing the functionality described herein.

The network 106 may include, or operate in conjunction with, an ad hoc network, an intranet, an extranet, a virtual private network (VPN), a local area network (LAN), a wireless network, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, Gobi™, Bluetooth™, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

In the example shown in FIG. 1, a user using the member-related client device 104 can establish a communication session with a health specialist (or provider) using the provider client device 102. While not shown in FIG. 1, a plurality of health specialists using a plurality of provider client devices 102 can be included in the system 100. The plurality of health specialists can include health providers that service different behavioral or mental healthcare needs. For example, the plurality of health specialists (or specialists) can include psychologists, psychiatrists, psychoanalysts, psychiatric nurses, psychotherapists, mental health counselors, family and marriage counselors, addiction counselors, etc. The member-related provider device 102 can load instructions from its memory or from a remote server and, at times, be a dedicated machine providing the functionality described herein related to a provider.

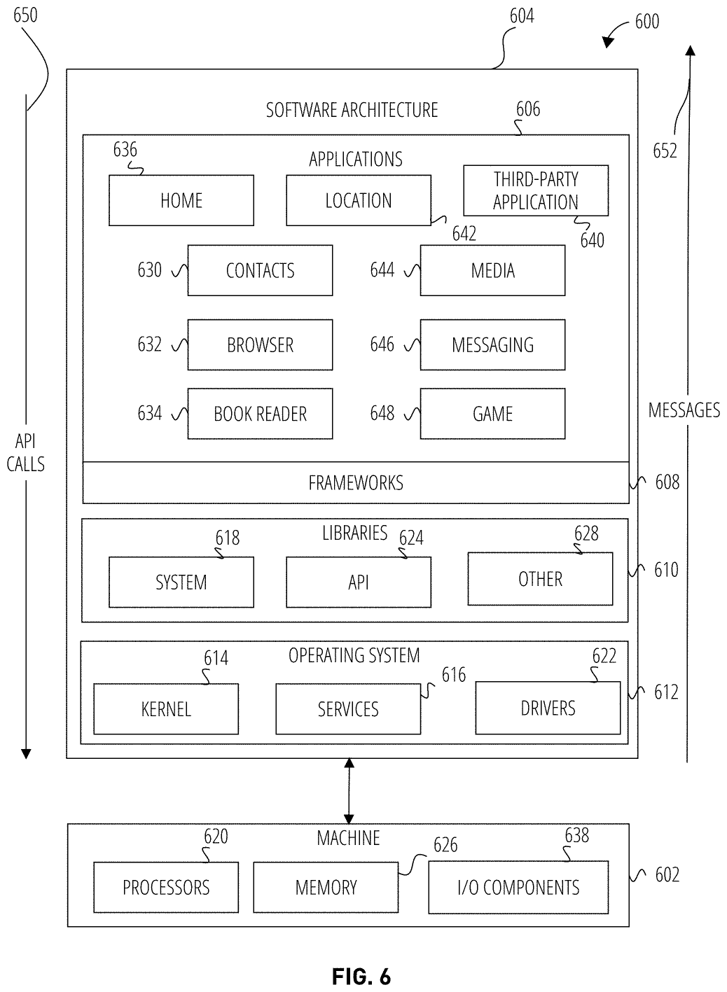

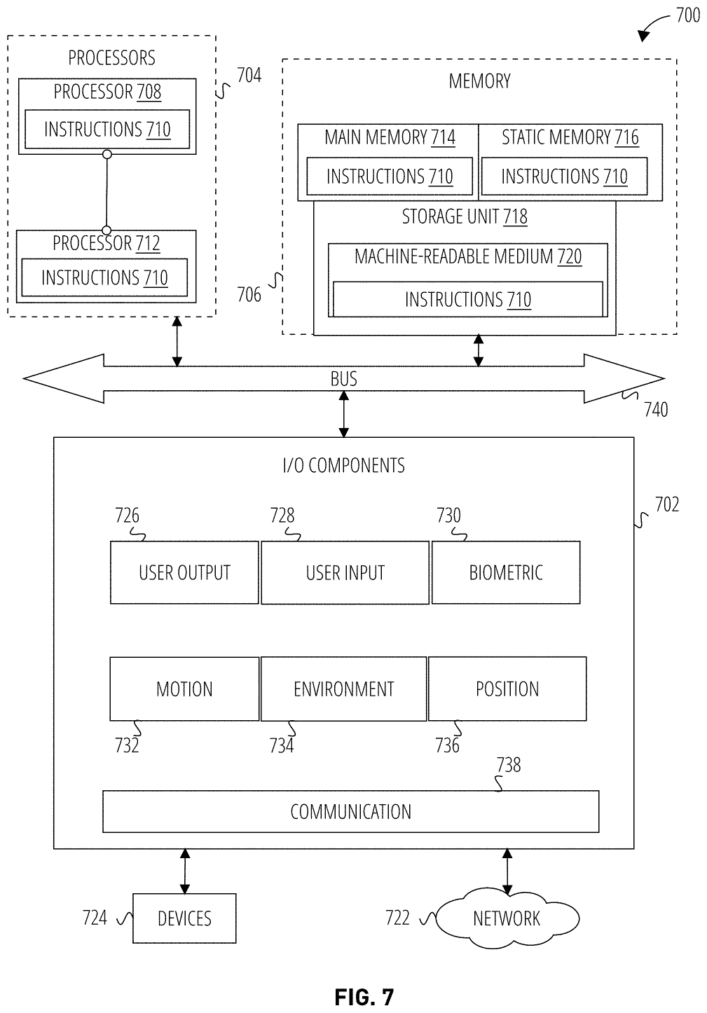

The customer service server system 112 in FIG. 1 includes a connection system 108. The customer service server system 112 can further include elements described with respect to FIG. 6 and FIG. 7, such as a processor and memory, having instructions stored thereon, that when executed by the processor, causes the processor to control the functions of the customer service server system 112. The connection system 108 can provide a secure connection between the member-related client device 104 and a selected provider client device 102, e.g., using a preferred connection from a plurality of different connections.

As shown in FIG. 1, the system 100 can also include a speech-to-text processor 110 that is communicatively coupled to the connection system 108. The speech-to-text processor 110 can be included in the connection system 108, the provider client device 102, the member-related client device 104, or any combination thereof.

### Connection System

In FIG. 1, the connection system 108 of the customer service server system 112 can quickly establish the communication session between a user and a provider when the user requests access to the provider. The connection system 108, using a machine learning model, can further optimize the fit or compatibility between the customer (or user) and a provider, by selecting a provider for the communication session based on factors including health care needs, customer preferences, customer satisfaction scores associated with the providers, the contracted rate associated with the providers, etc.

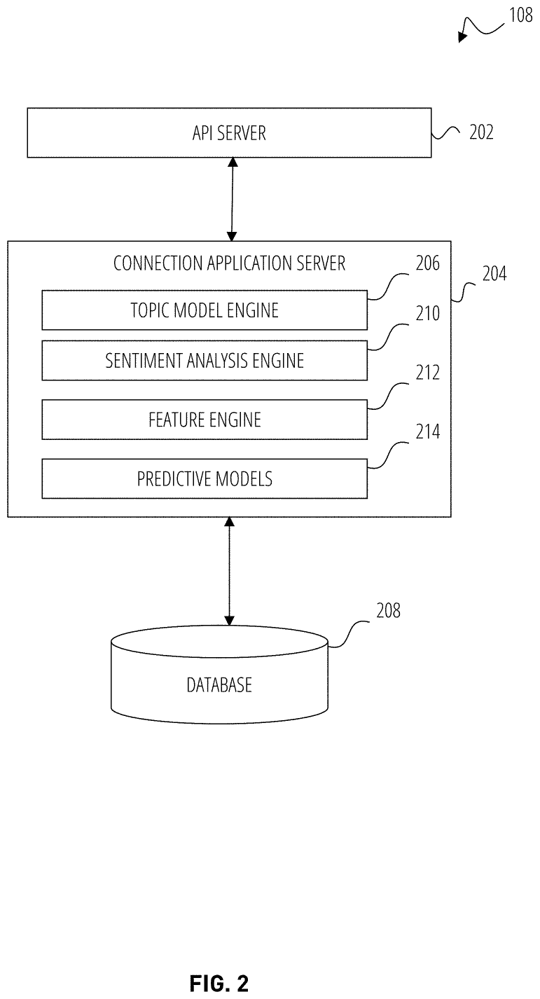

FIG. 2 is block diagram illustrating further details regarding the connection system 108, according to exemplary embodiments. The connection system 108 includes an API server 202 that is coupled to and provides a programmatic interface to a connection application server 204. For example, the connection application server 204, using the API server 202, receive the request from the member-related client device 104 for a communication session to be established with specialist (or provider) using a provider client device 102 and transmit a signal to the provider client device 102 to establish a communication session between the member-related client device 104 and a provider client device 102. The communication session can include, for example, a communication session via text (e.g., chat, direct messages, electronic text communications, etc.), audio (e.g., telephone call, VoIP call, etc.), or video (e.g., video teleconference via the Internet or cellular communication network).

FIG. 2 shows the connection application server 204, a topic model engine 206, a sentiment analysis engine 210, a feature engine 212 and a plurality of predictive models 214. Each of these devices can include instructions stored in a memory that can be loaded into processing circuitry, which in turn can access other data stored in a memory or request other data from a storage server.

For a user of the system 100, the topic model engine 206 can receive, from the database 208, the information that is reported or provided by the user during the on-boarding process or during the user's ongoing engagements with the system 100. For example, the reported information can include the user's name, address, age, identification number, telephone number, healthcare identification or information, health conditions, emergency contact information, etc. In one embodiment, the speech-to-text processor 110 can be used to process the information that is reported or provided by the user.

The topic model engine 206 can also receive, from the database 208, a history of previous provider notes pertaining to the user. Provider notes can include the provider's assessment of the user's mental conditions and physical conditions, the user's progress, the user's reaction to the medications prescribed, etc.

In one example, the topic model engine 206 analyzes prior provider notes from the provider client device 102 that is entered into customer service server system 112 as well as the information that is reported by the user and generates the representative keywords (e.g., suicide, domestic abuse, etc.).

Topic model engine 206 can thus summarize texts from prior notes and customer reported information into representative short words and phrases. In one example, the topic model engine 206 can include a built-in dictionary including list of terms such as suicide, overdose, abuse, domestic violence, alcoholism, depression, etc. to highlight these keywords. Since the topic model engine 206 generates and provides the keywords that represent prior provider notes and customer reported information to the selected specialist's provider client device 102, the selected provider is thus provided context and background for the communication session with the user. In one embodiment, the keywords generated by topic model engine 206 can also be used by the connection system 108 to select the specialist by specialty (e.g., suicide prevention, anxiety, insomnia, etc.) to be matched with the user such that the selection is made based on the specific needs of the user. For example, if the keywords for one user (or customer) is “anxiety” and “insomnia”, then only the specialists with training or experience in treating both anxiety and insomnia will be kept in the pool for matching. In one example, each specialist enrolled in the system 100, using the provider client devices 102, can select keywords such as “depression”, “anxiety”, etc. from a list of terms to highlight their area of expertise. Specialists can select all terms that align with their prior experience, training, and proficiency.

The sentiment analysis engine 210 can also receive the reported information from the user and the history of previous provider notes pertaining to the user from the database 208. The sentiment analysis engine 210 can use the reported information from the user and the history of previous provider notes pertaining to the user to generate a negative emotion score that is stored in the database 208.

The sentiment analysis engine 210 can convert unstructured text from customer (or user) reported information into a numerical score (e.g., normalized, binary (positive or negative), ranging from 0 to 1, or other scoring) to evaluate the level of positive or negative emotions from patients over time. For instance, the sentiment analysis engine 210 that receives the customer provided statement, “I feel very down, cannot stop thinking in bad ways”, can generate a negative emotion score of (“Negative”, 0.545); while the sentiment analysis engine 210 that receives the customer provided statement, “I hate everyone around including myself”, can generate a negative emotion score of (“Negative”, 0.852). While both statements are very negative, the latter statement has a higher level of negative emotion compared with the first one.

In an example, the connection system 108 can cause the negative emotion score to be displayed by the selected provider's provider client device 102 as a reference to quickly reflect patients' emotion dynamics. The instructions on the provider device 102 can organize its display to show the emotion score thereon with information regarding the patient. In an embodiment, the reported information from the user and the history of previous provider notes pertaining to the user can be processed by the sentiment analysis engine 210 to generate a plurality of standard data attributes such as the maximum negative emotion score over past three months, median negative emotion score over past three months, fluctuation of emotion (or negative emotion scores) over past three months, etc. Using the negative emotion score and the keywords, the quality and effectiveness of the conversation can be enhanced even though the customer and provider may be speaking for the first time.

While the present disclosure uses the negative emotion score, it is within the scope of the present disclosure to use a positive emotion score in a similar manner. For example, the provider device can invert a negative emotion score to a positive emotion score such that a trend display will show the trend line going up when the system scores the patient as increasing positivity over time. For example, the score can be normalized between a maximum (e.g., one) and a minimum (e.g., zero). When the system (e.g., sentiment analysis engine 210) uses a negative scoring, the maximum is the most negative score and the minimum is the least negative score. When the system (e.g., sentiment analysis engine 210) uses a positive scoring, the maximum is the most positive score and the minimum is the least positive score.

In FIG. 2, the feature engine 212 receives user data from the database 208 and the negative emotion score from the sentiment analysis engine 210. The user data can comprise claim history which includes medical data, pharmacy data, adherence data, costs data, and the prescriber information associated with the users, as well as demographic data such as age, gender, socio-economic conditions, etc.

Using the user data and the negative emotion score, the feature engine 212 can generate selected features associated with a selected target of a given condition or issue. Issues can include basis for a user to seek assistance from a provider through the system described herein. For example, in the case of depression or loneliness, depression progression can be defined as the target and in the case of anxiety, chronic utilization of prescription medication can be defined as the target. The selected features for each of these targets can include, for example, demographic features, the number of prescription claims in a given claim period, the number of claims before the given claim period, the number of prescription claims in an index period, the prescriber information, the adherence information, the pharmacy information, the cost information, or the user's claim history, etc.

The feature engine 212 can identify new features based on analysis of the data that is stored in the database. These new features can be associated with a given condition or issue. The feature engine 212 can identify new conditions or new issues.

Using the selected features, the plurality of predictive models 214 with different targets are trained and developed. The predictive models 214 can each be associated with different issues or conditions including depression, insomnia, anxiety, abuse, etc. For example, the predictive models 214 can include a depression model, an insomnia model, an anxiety model, an abuse model, etc. Each of the models receives the selected features associated with the respective condition and generates risk scores for each of the targets or conditions (depression, anxiety, insomnia, domestic violence or abuse). In an embodiment, when a customer requests a provider for a communication session, the provider, e.g., through the provider client device, will immediately get the risk scores and representative key words for the customer for background and context.

The predictive models 214 can be trained and refreshed with most recent data in the database 208 (e.g., most recent 12 month) every month to better satisfy emerging needs from customers. In an example, when the connection system 108 detects that the risk score generated by one of the predictive models 214 exceeds a threshold (e.g., 0.7 out of 1 during past three months), this risk score and associated condition or issue is tagged or flagged for this customer to be leveraged for matching with a specialist. The threshold can be predetermined or can be adjusted in the system based on the trends of a specific class of patients or historical data of patient's treatment data.

In an example, any customer associated with a risk score generated by one of the predictive models 214 that exceeds a threshold (e.g., greater than or equal to 0.7 during past 3 months) is tagged with the corresponding keywords. For example, if the risk scores per predictive models 214 during past 3 months for a customer are: depression—0.873, anxiety—0.154, insomnia—0.721, domestic abuse/self-harm—0.104, then the customer will be marked as “depression”, “insomnia” as keywords.

The connection application server 204 is communicatively coupled to the database 208, in which is stored data processed by the connection application server 204 to generate the risk scores and the key words, as further described herein. In one embodiment, the connection application server 204 includes a memory that stores instructions, when executed by a processor, causes processor to perform the operations of the connection application server 204, i.e., when loaded with the instructions the processor is a dedicated machine.

### Data Architecture

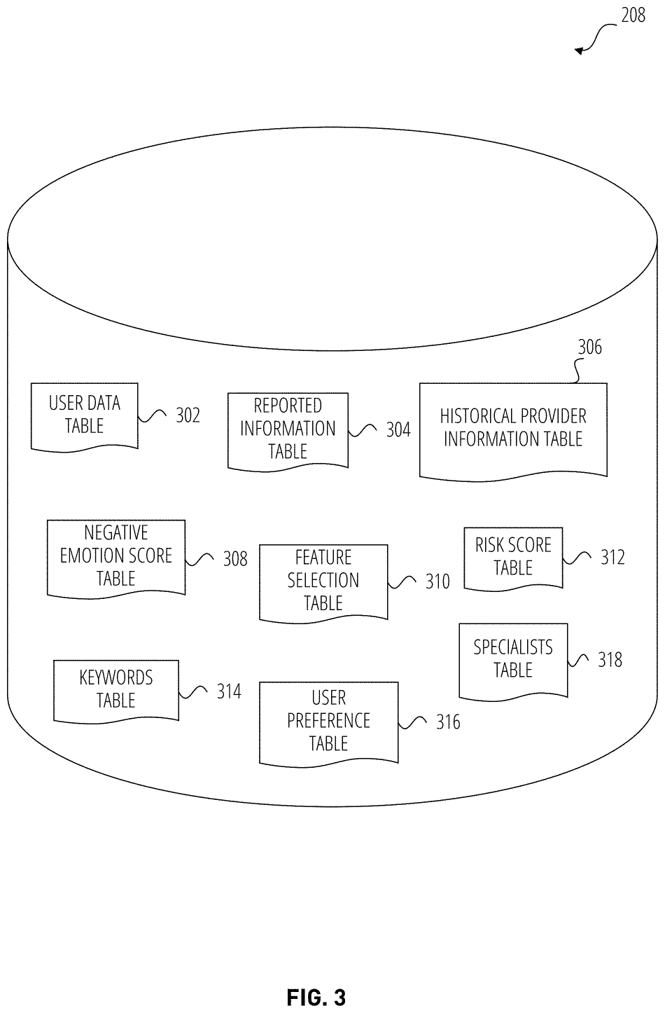

FIG. 3 is a schematic diagram illustrating data that is stored in the database 208 of the connection system 108, according to certain exemplary embodiments. While the content of the database 208 is shown to comprise a number of tables, the data could be stored in other types of data structures (e.g., as an object-oriented database). The database 208 includes a user data table 302, a reported information table 304, a historical provider information table 306, an emotion score table 308 (e.g., a negative emotion score table), a feature selection table 310, a risk score table 312, keywords table 314, user preference table 316, and a specialists table 318.

The user data table 302 stores user data for each of the users in the system 100. The user data can comprise claim history which includes medical and pharmacy data. The user data can include adherence data, costs data, and the prescriber information associated with the users. The user data can further comprise demographic data such as age, gender, socio-economic conditions, etc.

The reported information table 304 stores information that is reported or provided by each of the users in the system 100 during the onboarding process or during ongoing engagements or through the member client device 104. For example, the reported information can include the user's name, address, age, identification number, telephone number, healthcare identification or information, health conditions, emergency contact information, etc.

The historical provider information table 306 stores a history of previous provider notes pertaining to each of the users of the system 100. Provider notes can include the provider's assessment of the user's mental conditions and physical conditions, the user's progress, the user's reaction to the medications prescribed, etc.

The negative emotion score table 308 stores the negative emotion scores generated by the sentiment analysis engine 210 for the users of the system 100. The table 308 can also store aggregate emotion scores for classes of users, e.g., groupings of users.

The feature selection table 310 stores the features that are generated by the feature engine 212 for each of the users in the system 100.

The risk score table 312 stores the risk scores generated by the predictive models 214 for each of the conditions for each of the users of the system 100.

The keywords table 314 stores the keywords that are generated by the topic model engine 206. In one example, the topic model engine 206 using a natural language processing engine analyzes prior provider notes and the information reported by the users and extracts keywords (e.g., suicide, domestic abuse, etc.) that are stored in historical provider information table 306 in association with the users. The keywords can also represent the conditions or issues identified as described herein.

The user preference table 316 stores each of the users of the system's 100 preference (e.g., age, language, gender, etc.) when being matched with a provider or specialist.

The specialists table 318 stores data on the health specialists or providers in the system 100 such as the specialist's or provider's name, age, gender, languages spoken or written, location, medical or professional licenses, years of practice, customer satisfaction scores, etc. The specialists table 318 can also store the keywords that are associated with each of the health specialists. When new keywords are identified using the engines described herein, the system 100 will notify the provider of new keywords, e.g., through the provider client device 102, that the provider through their associated client device 102 will select to assign to their profile stored at the system 112, which selection the system will use to predict a match to potential users.

Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems, such as the systems described in FIG. 1 and/or FIG. 7.

Process of Establishing an Instant Communication Session with A Health Provider

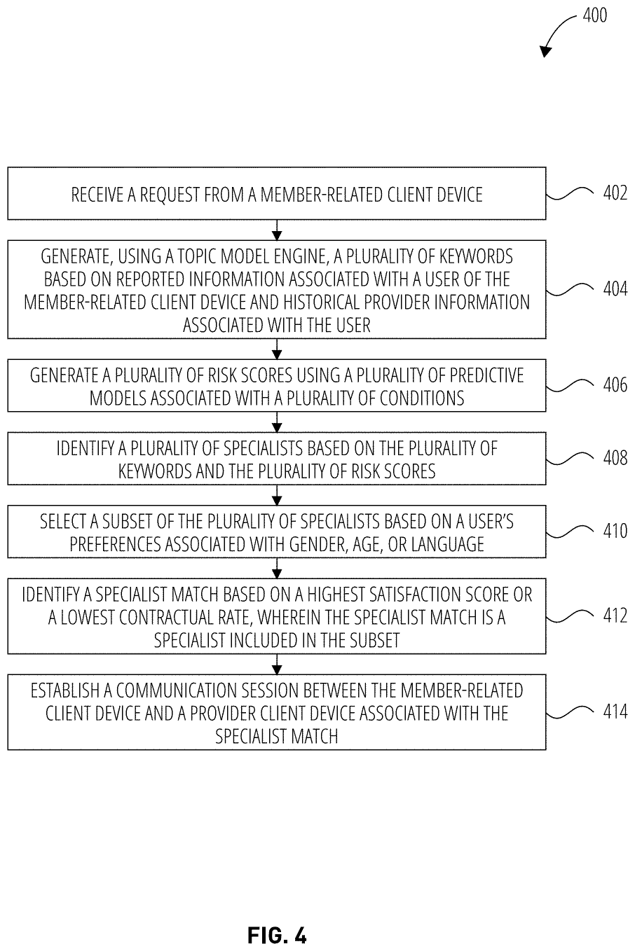

FIG. 4 is a flow diagram of an exemplary process of establishing an instant communication session with a health provider in accordance with various embodiments. In an embodiment, a processor (or circuitry dedicated to performing instructed tasks) included in the connection system 108 performs the process 400 or causes the connection system 108 to perform the process 400.

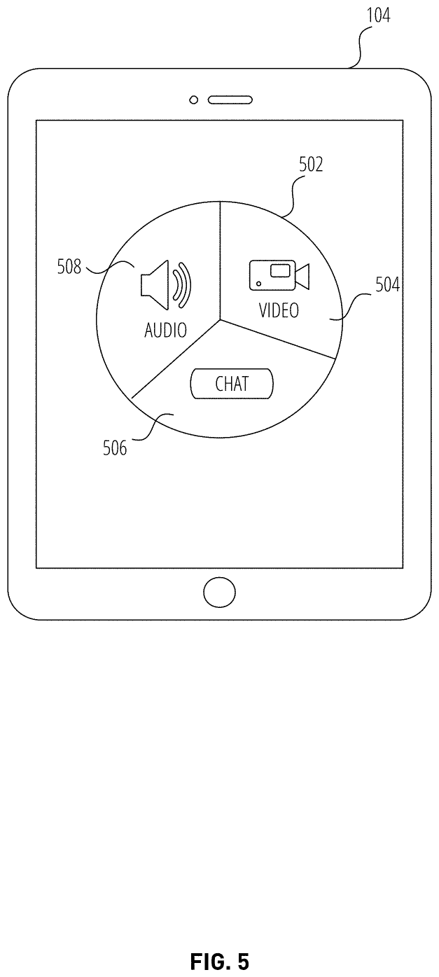

In operation 402, a processor receives a request from a member-related client device 104. For example, the request can be received in response to the user activating a selectable item 502 as shown in FIG. 5 being displayed on the member-related client device 104. FIG. 5 illustrates a user interface of a member-related client device 104 displaying a selectable item 502 in accordance with one embodiment. The selectable item 502 can include a plurality of selectable portions 504, 506, and 508. Each of the selectable portions 504, 506, and 508 can be associated with a plurality of communication preferences. As shown in FIG. 5, the selectable portion 504 is associated with video (e.g., video teleconference), the selectable portion 506 is associated with text communications (e.g., direct messages, chat, text messages, SMS, etc.), and the selectable portion 508 is associated with audio (e.g., telephone call, voice call via VoIP, etc.). In an example, the processor determines the communication preference that is associated with the request, and the communication session that is established between the member-related client device 104 and the provider client device 102 is based on the communication preference associated with the request. For example, the processor can determine the communication preference by determining the selectable portion that is selected by the user by engaging the member client device when activating the selectable item 502. The selectable portion that is selected is one of the plurality of selectable portions 504, 506, 508.

In operation 404, the processor generates, using a topic model engine 206, a plurality of keywords based on reported information associated with a user of the member-related client device 104 and historical provider information associated with the user.

The processor can analyze, using a sentiment analysis engine and natural language processing, the reported information associated with the user or the historical provider information associated with the user to generate unstructured text. The processor can convert the unstructured text into a negative emotion score that is a numerical score.

In an example, the processor can generate, using a feature engine, a feature selection based on data associated with the user and the negative emotion score.

The data associated with the user includes a claim history, adherence history, a prescriber associated with the user, cost information associated with the user, demographic information associated with the user, the reported information associated with the user, the historical provider information associated with the user, or any combination thereof.

The processor can use the feature selection to train a plurality of predictive models 214 that are associated with a plurality of conditions that includes, for example, depression, loneliness, anxiety, insomnia, domestic violence, abuse, etc.

In operation 406, the processor generates a plurality of risk scores using the plurality of predictive models associated with the plurality of conditions.

In operation 408, the processor identifies a plurality of specialists based on the plurality of keywords and the plurality of risk scores. In one example, the processor can cause the provider client devices 102 associated with the plurality of specialists to display the plurality of keywords and the plurality of risk scores.

In operation 410, the processor selects a subset of the plurality of specialists based on a user's preferences associated with gender, age, or language. In one embodiment, the processor selects the subset to include the specialists that match the user's language, gender, and age preferences. If no specialists match the user's language, gender, and age preferences, the processor selects the subset to include the user's language and gender preferences. If no specialists match the user's language and gender preferences, the processor selects the subset to include the user's language and age preferences. If no specialists match the user's language and age preferences, the processor selects the subset to include the user's language preferences. If no specialists match the user's language preferences, the processor selects the subset to include any available specialist.

In operation 412, the processor identifies a specialist match based on a highest satisfaction score or a lowest contractual rate. The specialist match can be a specialist included in the subset. In one embodiment, each specialist in the plurality of specialists is associated with a satisfaction score and a contractual rate. In this example, the processor can determine the highest satisfaction score associated with the specialists in the subset and the lowest contractual rate associated with the specialists in the subset.

In an embodiment, the processor can sort the providers in the subset by customer satisfaction scores. The processor can generate a high score group that includes specialists that are associated with the highest satisfaction score. In an example, the high score group is included in the subset. For instance, the high score group can be a subset of the subset where the high score group includes a number of specialists from the subset that are associated with the highest satisfaction score. In an embodiment, the processor can generate a high score group that includes specialists that are associated with the highest satisfaction score tier. The highest satisfaction score tier can include a range of satisfaction scores (e.g., 8.1 to 10 on a scale of 10).

In an example, the processor can send a request to connect and initiate the interaction with the user to the provider client devices 102 of the specialists in the subset, but the communication session is routed and established with the specialist with the high score group.

In an embodiment, the processor determines whether the high score group includes more than one specialist. In response to determining that the high score group does not include more than one specialist, the processor identifies the specialist match as the specialist included in the high score group. In response to determining that the high score group does include more than one specialist, the processor identifies the specialist match as a specialist in the high score group that is associated with the lowest contractual rate.

For instances where there are multiple providers in the high score group that accept request to connect and initiate the interaction, the selection of the specialist with whom to establish the communication session is based on the specialist that has a lower contracted rate. In an example, if the contracted rate is the same between multiple providers in the high score group, the connection is made based on the specialist who first accepted the request to connect and initiate the interaction.

In operation 414, the processor establishes a communication session between the member-related client device 104 and a provider client device 102 associated with the specialist match. In an embodiment, the processor causes the provider client device 102 to display the plurality of risk scores and the plurality of keywords.

The processor can determine one of the plurality of risk scores exceeds a set threshold or a predetermined threshold. In response to determining that one of the plurality of risk scores exceeds the threshold, the processor can cause an alert or flag associated with the user and the one of the plurality of risk scores to be generated by the provider client device 102.

The processor can receive a selection of a set of keywords from the provider client devices 102 and store the set of keywords in association with the specialists associated with the provider client devices 102. For example, the processor can receive a selection of a set of keywords from the provider client device 102 and store the set of keywords in association with the specialist match in the database 208.

Accordingly, the processor of the connection system 108, using a machine learning model, can further optimize the fit or compatibility between the user and a specialist, quickly establish the communication session with the specialist match via a one-touch activation of the selectable item 502, and further, provide the specialist with keywords and risk scores that provide the context and background on the user to better inform the specialist prior to or during the communication session.

### Software Architecture

FIG. 6 is a block diagram 600 illustrating a software architecture 604, which can be installed on any one or more of the devices described herein. The software architecture 604 is supported by hardware such as a machine 602 that includes processor circuitry 620, memory 626, and I/O components 638. In this example, the software architecture 604 can be conceptualized as a stack of layers, where each layer provides a particular functionality. The software architecture 604 includes layers such as an operating system 612, libraries 610, frameworks 608, and applications 606. Operationally, the applications 606 invoke API calls 650 through the software stack and receive messages 652 in response to the API calls 650.

The operating system 612 manages hardware resources and provides common services. The operating system 612 includes, for example, a kernel 614, services 616, and drivers 622. The kernel 614 acts as an abstraction layer between the hardware and the other software layers. For example, the kernel 614 provides memory management, processor management (e.g., scheduling), component management, networking, and security settings, among other functionality. The services 616 can provide other common services for the other software layers. The drivers 622 are responsible for controlling or interfacing with the underlying hardware. For instance, the drivers 622 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 610 provide a common low-level infrastructure used by the applications 606. The libraries 610 can include system libraries 618 (e.g., C standard library) that provide functions such as memory allocation functions, string manipulation functions, mathematic functions, and the like. In addition, the libraries 610 can include API libraries 624 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 610 can also include a wide variety of other libraries 628 to provide many other APIs to the applications 606.

The frameworks 608 provide a common high-level infrastructure that is used by the applications 606. For example, the frameworks 608 provide various graphical user interface (GUI) functions, high-level resource management, and high-level location services. The frameworks 608 can provide a broad spectrum of other APIs that can be used by the applications 606, some of which may be specific to a particular operating system or platform.

In an example, the applications 606 may include a home application 636, a contacts application 630, a browser application 632, a book reader application 634, a location application 642, a media application 644, a messaging application 646, a game application 648, and a broad assortment of other applications such as a third-party application 640. The applications 606 are programs that execute functions defined in the programs. Various programming languages can be employed to create one or more of the applications 606, structured in a variety of manners, such as object-oriented programming languages (e.g., Objective-C, Java, or C++) or procedural programming languages (e.g., C or assembly language). In a specific example, the third-party application 640 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 640 can invoke the API calls 650 provided by the operating system 612 to facilitate functionality described herein.

### Machine Architecture

FIG. 7 is a diagrammatic representation of the machine 700 within which instructions 710 (e.g., software, a program, an application, an applet, an app, or other executable code) for causing the machine 700 to perform any one or more of the methodologies discussed herein may be executed. For example, the instructions 710 may cause the machine 700 to execute any one or more of the methods described herein. The instructions 710 transform the general, non-programmed machine 700 into a particular machine 700 programmed to carry out the described and illustrated functions in the manner described. The machine 700 may operate as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 710, sequentially or otherwise, that specify actions to be taken by the machine 700. Further, while only a single machine 700 is illustrated, the term “machine” shall also be taken to include a collection of machines that individually or jointly execute the instructions 710 to perform any one or more of the methodologies discussed herein. The machine 700, for example, may comprise the provider client device 102 or any one of a number of server devices in member-related client device 104. In some examples, the machine 700 may also comprise both client and server systems, with certain operations of a particular method or algorithm being performed on the server-side and with certain operations of the particular method or algorithm being performed on the client-side.

The machine 700 may include processors 704, memory 706, and input/output I/O components 638, which may be configured to communicate with each other via a bus 740. In an example, the processors 704 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 708 and a processor 712 that execute the instructions 710. The term “processor” is intended to include multi-core processors that may comprise two or more independent processors (sometimes referred to as “cores”) that may execute instructions contemporaneously. Although FIG. 7 shows multiple processors 704, the machine 700 may include a single processor with a single-core, a single processor with multiple cores (e.g., a multi-core processor), multiple processors with a single core, multiple processors with multiples cores, or any combination thereof.

The memory 706 includes a main memory 714, a static memory 716, and a storage unit 718, both accessible to the processors 704 via the bus 740. The main memory 706, the static memory 716, and storage unit 718 store the instructions 710 embodying any one or more of the methodologies or functions described herein. The instructions 710 may also reside, completely or partially, within the main memory 714, within the static memory 716, within machine-readable medium 720 within the storage unit 718, within at least one of the processors 704 (e.g., within the Processor's cache memory), or any suitable combination thereof, during execution thereof by the machine 700.

The I/O components 702 may include a wide variety of components to receive input, provide output, produce output, transmit information, exchange information, capture measurements, and so on. The specific I/O components 702 that are included in a particular machine will depend on the type of machine. For example, portable machines such as mobile phones may include a touch input device or other such input mechanisms, while a headless server machine will likely not include such a touch input device. It will be appreciated that the I/O components 702 may include many other components that are not shown in FIG. 7. In various examples, the I/O components 702 may include user output components 726 and user input components 728. The user output components 726 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 728 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 702 may include biometric components 730, motion components 732, environmental components 734, or position components 736, among a wide array of other components. For example, the biometric components 730 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 732 include acceleration sensor components (e.g., accelerometer), gravitation sensor components, rotation sensor components (e.g., gyroscope).

The environmental components 734 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

The position components 736 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 702 further include communication components 738 operable to couple the machine 700 to a network 722 or devices 724 via respective coupling or connections. For example, the communication components 738 may include a network interface component or another suitable device to interface with the network 722. In further examples, the communication components 738 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 724 may be another machine or any of a wide variety of peripheral devices (e.g., a peripheral device coupled via a USB).

Moreover, the communication components 738 may detect identifiers or include components operable to detect identifiers. For example, the communication components 738 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 738, such as location via Internet Protocol (IP) geolocation, location via Wi-Fi® signal triangulation, location via detecting an NFC beacon signal that may indicate a particular location, and so forth.

The various memories (e.g., main memory 714, static memory 716, and memory of the processors 704) and storage unit 718 may store one or more sets of instructions and data structures (e.g., software) embodying or used by any one or more of the methodologies or functions described herein. These instructions (e.g., the instructions 710), when executed by processors 704, cause various operations to implement the disclosed examples.

The instructions 710 may be transmitted or received over the network 722, using a transmission medium, via a network interface device (e.g., a network interface component included in the communication components 738) and using any one of several well-known transfer protocols (e.g., hypertext transfer protocol (HTTP)). Similarly, the instructions 710 may be transmitted or received using a transmission medium via a coupling (e.g., a peer-to-peer coupling) to the devices 724.

### Glossary

“Carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

“Client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network. However, the client device when loaded with instructions is a dedicated machine.

“Communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“Computer-readable storage medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A system comprising:
a processor; and
a memory component having instructions stored thereon, when executed by the processor, causes the processor to perform operations comprising:
receiving from a member-related client device a request,
generating, using a topic model engine, a plurality of keywords based on reported information associated with a user of the member-related client device and historical provider information associated with the user;
generating a plurality of risk scores using a plurality of predictive models associated with a plurality of conditions;
identifying a plurality of specialists based on the plurality of keywords and the plurality of risk scores;
selecting a subset of the plurality of specialists based on a user's preferences associated with gender, age, or language;
identifying a specialist match based on a highest satisfaction score or a lowest contractual rate, wherein the specialist match is a specialist included in the subset; and
establishing a communication session between the member-related client device and a provider client device associated with the specialist match.

2. The system of claim 1, wherein the processor to perform operations further comprising:
determining the highest satisfaction score in the subset, wherein each specialist in the plurality of specialists is associated with a satisfaction score.

3. The system of claim 2, wherein the processor to perform operations further comprising:
determining the lowest contractual rate in the subset, wherein each specialist in the plurality of specialists is associated with a contractual rate.

4. The system of claim 3, wherein identifying a specialist match further comprises:
generating a high score group that includes specialists that are associated with the highest satisfaction score, wherein the high score group is included in the subset; and
determining whether the high score group includes more than one specialist.

5. The system of claim 4, wherein identifying a specialist match further comprises:
in response to determining that the high score group does not include more than one specialist,
identifying the specialist match as the specialist included in the high score group.

6. The system of claim 4, wherein identifying a specialist match further comprises:
in response to determining that the high score group does include more than one specialist,
identifying the specialist match as a specialist in the high score group that is associated with the lowest contractual rate.

7. The system of claim 1, wherein the request is received in response to the user activating a selectable item being displayed on the member-related client device.

8. The system of claim 7, wherein the selectable item comprises a plurality of selectable portions associated with a plurality of communication preferences, wherein the communication preferences comprise text, audio, or video.

9. The system of claim 8, wherein the processor to perform operations further comprising:
determining a first communication preference associated with the request, wherein the plurality of communication preferences comprises the first communication preference,
wherein the communication session between the member-related client device and the provider client device is based on the first communication preference.

10. The system of claim 9, wherein determining the first communication preference comprises:
determining a first selectable portion selected by the user, wherein the first selectable portion is associated with the first communication preference, wherein the plurality of selectable portions comprises the first selectable portion.

11. The system of claim 1, wherein the processor to perform operations further comprising:
analyzing, using a sentiment analysis engine and natural language processing, the reported information associated with the user or the historical provider information associated with the user to generate unstructured text; and
converting the unstructured text into a negative emotion score that is a numerical score.

12. The system of claim 11, wherein the processor to perform operations further comprising:
generating, using a feature engine, a feature selection based on data associated with the user and the negative emotion score; and
training the plurality of predictive models using the feature selection.

13. The system of claim 12, wherein the data associated with the user comprises a claim history, adherence history, a prescriber associated with the user, cost information associated with the user, demographic information associated with the user, the reported information associated with the user, the historical provider information associated with the user, or any combination thereof.

14. The system of claim 1, wherein the plurality of conditions includes depression, loneliness, anxiety, insomnia, domestic violence, or abuse.

15. The system of claim 1, wherein the processor to perform operations further comprising:
causing the provider client device to display the plurality of risk scores and the plurality of keywords.

16. The system of claim 15, wherein the processor to perform operations further comprising:
determining whether one of the plurality of risk scores exceeds a predetermined threshold.

17. The system of claim 16, wherein the processor to perform operations further comprising:
in response to determining that one of the plurality of risk scores exceeds the predetermined threshold,
causing an alert or flag associated with the user and the one of the plurality of risk scores to be generated by the provider client device.

18. The system of claim 1, wherein the processor to perform operations further comprising:
receiving a selection of a set of keywords from the provider client device; and
storing the set of keywords in association with the specialist match.

19. A method comprising:
receiving, by a processor, a request from a member-related client device;
generating, using a topic model engine, a plurality of keywords based on reported information associated with a user of the member-related client device and historical provider information associated with the user;
generating a plurality of risk scores using a plurality of predictive models associated with a plurality of conditions;
identifying a plurality of specialists based on the plurality of keywords and the plurality of risk scores;
selecting a subset of the plurality of specialists based on a user's preferences associated with gender, age, or language;
identifying a specialist match based on a highest satisfaction score or a lowest contractual rate, wherein the specialist match is a specialist included in the subset; and
establishing a communication session between the member-related client device and a provider client device associated with the specialist match.

20. A non-transitory computer-readable storage medium, having stored thereon, instructions, when executed by a processor, causes the processor to perform operations comprising:
receiving from a member-related client device a request;
generating, using a topic model engine, a plurality of keywords based on reported information associated with a user of the member-related client device and historical provider information associated with the user;
generating a plurality of risk scores using a plurality of predictive models associated with a plurality of conditions;
identifying a plurality of specialists based on the plurality of keywords and the plurality of risk scores;
selecting a subset of the plurality of specialists based on a user's preferences associated with gender, age, or language;
identifying a specialist match based on a highest satisfaction score or a lowest contractual rate, wherein the specialist match is a specialist included in the subset; and
establishing a communication session between the member-related client device and a provider client device associated with the specialist match.
